(19) United States
(12) Patent Application Publication
(10) Pub. No.: US 2022/0202435 A1
(43) Pub. Date: Jun. 30, 2022

(54) METRIC DISSECTION FORCEPS

(71) Applicant: Beatriz SIMANCAS SAN MARTÍN, Madrid (ES)

(72) Inventor: Beatriz SIMANCAS SAN MARTÍN, Madrid (ES)

(21) Appl. No.: 17/604,118

(22) PCT Filed: Dec. 4, 2019
(86) PCT No.: PCT/ES2019/070829

§ 371 Date: Oct. 15, 2021

(30) Foreign Application Priority Data
Apr. 17, 2019 (ES) U201900206

(51) Int. Cl. A61B 17/30; A61B 90/00

(52) U.S. Cl. CPC A61B 17/30; A61B 2090/062; A61B 90/06

(57) ABSTRACT

The present modification describes a surgical instrument which introduces a metric scale from the distal area of the forceps up to 5 cm proximal, allowing numerous uses, among which can be highlighted the measurement of the depth of ulcerous lesions following a plane perpendicular to the skin.

### TECHNICAL FIELD

Health Subsection. Surgical instrument

### BACKGROUND ART

The medical or surgical forceps, currently called dissecting forceps (1), is a metal instrument with two arms or branches which serves to bring closer, grab, grip, draw nearer or compress during distinct medical procedures such as surgery, healing of ulcers, removal of nonviable tissues, anatomic dissecting and other activities which require fine handling.

They have remote historical precedents which evolved slowly, using manual compression from their inception until the 18th century. Afterwards, clamp rings in the handle were introduced, thereby obtaining continuous pressure.

The forceps can be toothed or non-toothed. Non-toothed forceps will normally have a ridge on their inner surface and smooth tips, however, toothed forceps display tips similar to teeth which fit into each other on either side. Firm grip is obtained on heavy tissue and skin.

In the state of the art, European patent EP2391283 shows a microsurgery clamp, in particular a microincision capsulorhexis clamp, comprising a proximal handling portion (1) or handle including two branches or arms (1a, 1b) interconnected at one of the ends thereof, so as to be suitable for being approached or separated elastically, and a distal clamping portion (2) consisting of two narrow and thin clamping blades (2a, 2b) connected, respectively, to the distal ends of the handling branches (1a, 1b) and oriented laterally with respect to the distal end of each of the clamping blades (2a, 2b) being curved in a direction opposed to the handle (1), such that they constitute a clamping tip (3a, 3b), characterized in that the clamping blades (2a, 2b) are asymmetrical and oriented in a direction or plane (P1) corresponding to the direction or plane of approach and separation of the handling branches (1a, 1b), the aforementioned clamping blades (2a, 2b) being arranged above each other, relative to a vertical position of said handle (1), such that the approach or separation of said handling branches (1a, 1b) cause the corresponding approach or separation of the aforementioned clamping tips (3a, 3b) forming the jaws of the clamp.

As can be observed in the state of the art, no forceps, neither toothed nor non-toothed, has been shown to incorporate a system for measuring at the tips, such as a metric scale, by means of which the present invention allows quantitative measurements to be made of the depth in the area of intervention. The metric dissecting forceps presented here is essentially designed for the millimetric measurement of the depth of chronic ulcers following a plane perpendicular to the surface.

### DESCRIPTION OF THE INVENTION

According to the CONUEI (National Conference for Consensus on Lower Limb Ulcers), ulcers of the lower limbs are defined as a spontaneous or accidental lesion in the lower limb, hose aetiology can refer to a pathological process, systemic or of the extremity, and which does not heal in the expected time period. Moreover, this consensus defines the classification of these lesions according to their morphology (depth and tissue structure).

Focusing on depth for the classification of the ulcer, this consensus refers to the degree of the lesion to the tissue, constituting a clinical and therapeutic reference point, both of diagnostic as well as prognostic for measurement of areas and diameters, such as VISITRAK and others, for carrying out studies of new therapies and dressing. Nevertheless, there is no clinical method for measuring depth other than visual estimation.

Unfortunately, the human eye is very subjective and can vary from one professional to another. Thus, the metric dissecting forceps provides a fast, easy and economical means of QUANTIFYING the evolution and improvement of lesions, especially those chronic lesions which seem to heal with respect to clinical signs and diameters of the lesion, but where the depth and regeneration of the deeper tissues are found to be altered due to various causes and associated risk factors.

The idea of modifying this type of dissecting forceps came about thanks to years of experience and the need for correct handling of certain pathologies, such as ulcers in distinct parts of the body which reach deep planes.

The metric dissecting forceps is designed for the millimetric measurement of the depth of chronic ulcers following a plane perpendicular to the surface of the lesion for the precise the proper handling of complex lesions such as Diabetic Foot Ulcers and others, allowing quantification of the volume with respect to dimensions, of depth as well as diameter, of the lesions which are being treated and evaluated by various health professionals and thus eliminating subjectivity.

### DESCRIPTION OF DRAWINGS

To complete the present description and with the object of improving the understanding of the characteristics of the invention, a set of drawings has been included as an integral part of the description, where the following has been represented:

The design of this instrument has as its main objective the quantitative measurement of depth, so that, at the most distal area of the forceps, it should present a scale perpendicular to the central axis of the forceps (2), each one assigned a measure of length corresponding to the International System of Units (SI).

FIG. 1/4 shows an oblique view of the straight dissecting forceps according to the present invention without the incorporated modification.

FIGS. 2/4 and 4/4 show, respectively, a toothless and a blunt-tip toothed straight dissecting forceps with the modification incorporated, displaying in detail the present invention for greater clarity.

FIG. 3/4 shows an oblique view of the ADSON forceps with the modification incorporated.

### BEST MODE FOR CARRYING OUT THE INVENTION

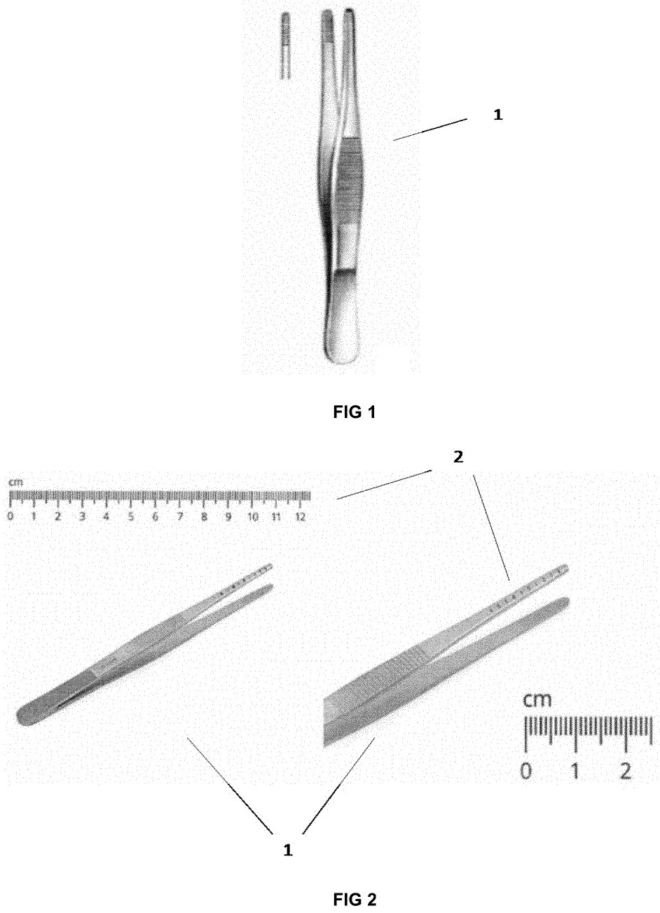

The following describes the modification of the invention, referring to the attached figures, specifically FIG. 1/4, showing the toothless straight dissecting forceps of approximately 14 cm with finely serrated tips which guarantee an excellent grip on the skin and the subcutaneous tissue, as base model on which to make the modification consisting of two metal arms joined at a vertex, angled at between 10° y 20° in an ergonomic fashion, with a blunt tip at the most distal area, to be used to handle deep tissues without damaging healthy tissue, as the object is not intended for cutting or puncturing.

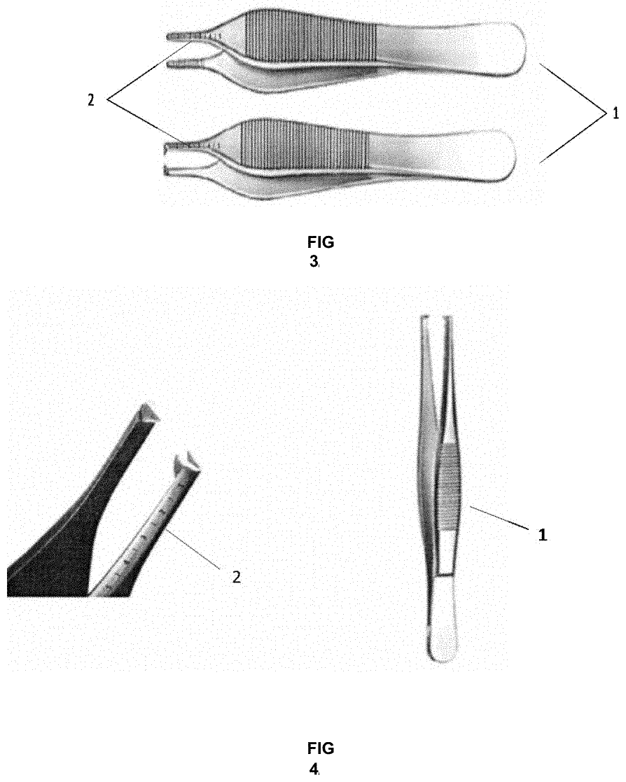

FIG. 2/4 shows a non-toothed straight dissecting forceps of approximately 14 cm ,with blunt tip and finely serrated tips (essential for functions such as measuring, debriding deep tissues, etc.) to which a metric scale is incorporated in the most distal area.

FIG. 3/4 shows another model of dissecting forceps, with a narrow tip, called the ADSON dissecting forceps, of 12 cm, toothed and toothless, which allows the measurement of lesions of lesser diameter. The modification in this type of forceps would also be useful due, on many occasions, to the small size of the lesion regardless of the depth, so that fine handling is required.

FIG. 4/4 shows another model of dissecting forceps, the Dissecting Forceps Standard Pattern 1:2 teeth—14 cm in length, which displays a tooth on each arm of the forceps at its most distal area, which is very useful for the elimination of deep devitalized tissue.

In FIGS. 2, 3, and 4, the modification of the aforementioned forceps is achieved from the most distal area by insertion of a metric scale of up to 5 cm proximal. In this way, by adding pressure to the arms of the forceps and joining both blunt tips, a form of measurement is created somewhat similar to a ruler graduated in millimeters or centimeters to measure lengths and distances.

1. Dissecting forceps (1) for the manipulation of human tissues characterized because it incorporates two metallic tips joined by a vertex and angled between 10° and 20° with blunt tips on the most distal zone, where a millimetric scale up to 5 cm proximal is added, such that upon putting pressure on the branches of the forceps and bringing both blunt tips together, an aid to measurement is created, in the form of a scale divided into millimeters or centimeters with which to quantify longitude and distance.

2. Dissecting forceps (1) in accordance with claim 1, characterized by having a narrow tip, non-toothed, for the assessment and manipulation of tunneling of lesser diameter.

3. Dissecting forceps (1) in accordance with claim 1, characterized by having a narrow tip, toothed, for the assessment and manipulation of tunneling of lesser diameter.

4. Dissecting forceps (1) in accordance with claim 1, characterized by an operative tooth in its most distal area in each branch of the forceps.

5. Dissecting forceps (1) in accordance with claim 1, whereas the clamp (1) has a length of 12 cm.

6. Dissecting forceps (1) in accordance with claim 1, whereas the clamp (1) has a length of 14 cm.
